(19) United States
(12) Patent Application Publication
(10) Pub. No.: US 2008/0199550 A1
(43) Pub. Date: Aug. 21, 2008

(54) Novel Anticancer Agent, Methods for Obtaining the Same and Pharmaceutical Compositions Thereof

(76) Inventors: Shanker Kumar Mitra, Bangalore (IN); Ekta Saxena, Bangalore (IN); Mallikarjun Narayan Dixit, Bangalore (IN); Venkanna Babu Uddagiri, Bangalore (IN); Venkata Ranganna Marikunte, Bangalore (IN); Shivamurthaiah Arun Mathad, Bangalore (IN); Sunil Vaikunth Shanbhag, Bangalore (IN)

Correspondence Address: DARBY & DARBY P.C., P.O. BOX 770, Church Street Station, New York, NY, 10008-0770, US

(21) Appl. No.: 11/917,269

(22) PCT Filed: Jun. 16, 2005
(86) PCT No.: PCT/IN05/00201

§ 371 Date: Feb. 29, 2008

(51) Int. Cl. A61K 36/18; A61K 31/343; A61P 35/00

(52) U.S. Cl. 424/778; 424/725; 514/468

(57) ABSTRACT

Disclosed herein a herbal anticancer agent comprising the extract of plant Sphaeranthus indicus or group of compounds obtained from the plant Sphaeranthus indicus. The present invention also discloses a pharmaceutical composition comprising the said agent, methods for preparing the composition and methods of treating all kinds of cancer in mammals including human beings. The methods of making the plant extract, methods for obtaining the active constituents are also dealt with.

### FIELD OF THE INVENTION

This invention, in general, relates to novel herbal anticancer agents. More specifically, but without restriction to the particular embodiments hereinafter described in accordance with the best mode of practice, this invention provides a herbal extract of plant Sphaeranthus indicus or group of compounds preferably sesquiterpenoids obtained from the extract of the plant Sphaeranthus indicus and pharmaceutical compositions thereof for effective treatment of cancer in mammals including human beings.

### BACKGROUND OF THE INVENTION

Cancers are uncontrolled cell proliferations that result from the accumulation of genetic changes in cells endowed with proliferative potential. The malignant cells progress to aggressive invasive and metastatic stages with tumor formation, bleeding, susceptibility to infections and wide spread dissimination throughout the body.

It is growing public health concern worldwide with new incidence of six million cases every year and cancer is second only to heart diseases as a cause of death in western countries.

Treatment of cancer has been the main objective of research and development in pharmaceutical industries for the last three decades. As a result, many approaches to cancer therapy have been discovered and investigated. Cancer therapy can involve use of multiple treatment methods including surgical excision, radiation therapy, chemotherapy, biological therapy, heat therapy and alternative therapy. The use of a particular therapy to treat a given malignancy depends on the nature and location and type of malignancy. Chemotherapy and radiotherapy are often used in combination.

Chemotherapy has been known to produce long term remission in patients with Hodgkin's Disease, testicular cancer, acute lymphocytic and myelogenous leukemia. It is often involves the use of combinations of chemotherapeutic agents.

The current FDA approved cancer drugs e.g. tamoxifen, cyclophosphamide, cisplatin, doxorubin etc. are unfortunately not totally effective and also cause many side effects for example uterine cancer in the case of tamoxifen.

In the last three decades, there has been remarkable increase in the usage of herbal medicines for various diseases. Discovery of Paclitaxel, Vincristine and Vinblastin from herbs as anticancer agents has certainly given momentum to herbal medicines as safe and effective in cancer chemotherapy. Unfortunately all these compounds are associated with some side effects such as alopecia, bone marrow depression, chest pain, bradycardia, hypersensitivity and altered liver function etc.

To overcome the challenges described above, major research activities have been directed towards developing new natural products which are in turn aimed at developing a formulation to have wide spectrum of anticancer property that is effective in all major cancer cell lines. The present invention is also aimed at safe and effective treatments for cancer preferably to treat all types of tumors for example breast cancer and other types of cancers like colon, cervix, neurobalstoma, liver, skin and leukemia.

### PRIOR ART

U.S. Pat. No. 6,811,795 to Yigzaw discloses a pharmaceutical composition comprising Glinus lotoides, Ruta chalepensis, Hagenia abyssinica etc. for treating cancer and a process for its preparation.

U.S. Pat. No. 6,812,258 to Bessette, at al. discloses a method of composition using plant essential oils for treating cancer.

U.S. Pat. No. 6,787,161 to Aylward teaches a method of processing anticancer compounds from Euphorbia species.

U.S. Pat. No. 6,780,441 to Solanki discloses a composition of eleven herbs treating cancer.

U.S. Pat. No. 6,649,650 to Rao, et al. discloses a herbal composition of lignans from Cedrus deodra exhibiting anticancer activities.

U.S. Pat. No. 6,649,185 to Solanki teaches the preparation of herbal composition comprising seven herbs for the treatment of cancer in particular to haematological malignancies.

U.S. Pat. No. 6,395,279 to Empie at al. discloses a method of preparing phytochemicals such as isoflavones for the treatment of cancer.

U.S. Pat. No. 6,790,464 to Kuok et al. discloses a herbal composition for prevention or treatment of prostate disorders and symptoms thereof including prostates, benign prostate hyperplasia and prostatic carcinoma.

U.S. Pat. No. 6,586,016 to Tsai et al. discloses a Chinese herbal composition, ST 188L for prevention and treatment of cancers and infectious diseases. The composition comprises Ecchinops grijissii, Cirsium Segetum Bge, Solanum indicum Linn, Lonicerae flos and Ziziphi fructus.

U.S. Pat. No. 6,565,897 to Selvaraj teaches the use of Nerium species extract and its pharmaceutical compositions for cell proliferative and immune deficient diseases in mammals including cancers and AIDS, respectively.

U.S. Pat. No. 6,521,271 to Phan discloses the method of treatment for skin conditions using turmeric extract

Sphaeranthus indicus has been reported to contain methyl chavicol, α-ionone, δ-cadinene, p-methoxycinnamaldehyde as major constituents and α-terpinene, citral geraniol, geranyl acetate, β-ionone, sphaerene, indicusene and sphaeranthol as minor constituents of essential oil. (Perfum. Essent. Oil Record. 1959,50,765; Chem. Abstr. 1960, 54, 7980g)

A new eudesmenolide, 7α-hydroxyeudesm-4en-6,12-olide, its β-isomer, dihydrolactone, a new sesquiterpene acid 2-hydroxycostic acid, β-eudesmol and illicic acid have been isolated from the plant Sphaeranthus indicus (Indian J Chem. 1986, 25B, 233; J. Chem. Soc. Perkin 1 1988, 157; J. Chem. Res. Synop. 1989, 68; Chem. Abstr. 1989, 111, 130706f).

Another three new eudesmanolides have been isolated and characterized from the flowers of the plant Sphaeranthus indicus (J. Nat. Prod. 1991, 54, 882)

### SUMMARY OF THE INVENTION

In accordance with the principal aspect of the present invention, there is provided a novel herbal anticancer agent comprising herbal extract of plant Sphaeranthus indicus or group of compounds preferably sesquiterpenoids obtained from the same.

In accordance with the other aspect of the present invention, there is provided a novel pharmaceutical composition comprising a therapeutically effective amount of extract of plant Sphaeranthus indicus or a group of compounds preferably sesquiterpenoids obtained from the same and pharmaceutically acceptable carrier or otherwise and using the same for effective treatment of all types of cancers in mammals including human beings.

In accordance with another aspect of the present invention, there is provided a novel pharmaceutical composition comprising a therapeutically effective amount of a group of compounds preferably sesquiterpenoids more preferably sesquiterpene lactones obtained from the plant Sphaeranthus indicus and pharmaceutically acceptable carrier or otherwise and using the same for effective treatment of all types of cancers in mammals including human beings.

In accordance with yet another aspect of the present invention, there is provided a novel pharmaceutical composition comprising a therapeutically effective amount of sesquiterpene lactones preferably 7-hydroxyeudesm-4-en-6,12-olide #STR#1 or any derivative of the said #STR#1 obtained from the plant Sphaeranthus indicus and pharmaceutically acceptable carrier or otherwise and using the same for effective treatment of all types of cancers in mammals including human beings.

In accordance with one preferred embodiment of the present invention, there is provided a method for extraction of the active constituents from Sphaeranthus indicus, identification and characterization of the same and preparing a pharmaceutical formulation employing the same with pharmaceutically acceptable carrier or otherwise for effective treatment of all types of cancers in mammals including human beings.

In one preferred embodiment, there is provided a herbal extract of plant Sphaeranthus indicus for the preparation of pharmaceutical formulation for cancer treatment, wherein the extract is prepared by all parts of said herb and preferably its aerial parts.

In another preferred embodiment, there is provided a herbal extract of plant Sphaeranthus indicus for the preparation of pharmaceutical formulation for cancer treatment, wherein the extract is prepared by all parts of said herb and preferably its flowers.

In still another preferred embodiment, there is provided a method for obtaining the plant extracts and constituents thereof, the method comprising extraction employing a conventional technique of percolation or hot soxhalation to provide a herbal extract of the plant Sphaeranthus indicus. Further, filtering the plant extract, concentrating the plant extract to dryness on rotatory evaporator or on a steam bath at optimum temperature and producing an active constituent #STR#1 from the herbal extract.

In yet another preferred embodiment, there is provided a herbal anticancer composition comprising a therapeutically effective amount of extracts of plant Sphaeranthus indicus comprising alkaloids, monoterpenes, sesquiterpenes, sesquiterpene lactones, sesquiterpene lactone glycosides, diterpenes, triterpenoids, fatty acids esters, hydrocarbons, amino acids etc as one of the active constituents.

In yet another preferred embodiment, there is provided a herbal anticancer composition effective against all types of cancers containing a therapeutically effective amount of sesquiterpene lactone and/or 7-hydroxyeudesm-4-en-6,12-olide and/or a derivative and/or extract of plant Sphaeranthus indicus in a pharmaceutically acceptable carrier wherein the composition is in oral and/or injectable (i.v.) form.

In another preferred embodiment, there is provided a herbal anticancer composition containing a therapeutically effective amount of active ingredient of Sphaeranthus indicus in an amount of 2 mg to 30 mg and pharmaceutically acceptable carrier(s) comprising Cremophor ELP (Polyoxyl-35-Casteroil purified) (33.3 mg to 500 mg), Benzyl alcohol (20 mg) and Water for Injection IP (qs) per 1 ml of injectable dosage form.

In another preferred embodiment, there is provided a herbal anticancer composition containing a therapeutically effective amount of active ingredient of Sphaeranthus indicus in an amount of 2 mg to 30 mg and pharmaceutically acceptable carrier(s) comprising Cremophor ELP (Polyoxyl-35-Casteroil purified) (33.3 mg to 500 mg), and Absolute alcohol IP (qs) per 1 ml of injectable dosage form.

In another preferred embodiment, there is provided a herbal anticancer composition comprising making granules containing a therapeutically effective amount of active ingredient of Sphaeranthus indicus in an amount of 50 mg to 500 mg and pharmaceutically acceptable carrier(s) comprising MCCP IP (224.0 mg to 574.0 mg), Pregelatinized starch IP (10.0 mg), Croscarmellose sodium BP (10.0 mg), Crosspovidone XL USP (2.0 mg), Colloidal silicon dioxide IP/USP (2.0 mg) and Magnesium stearate IP (2.0 mg) and DM Water (qs) per 50 to 500 mg tablet dosage form.

In another preferred embodiment, there is provided a herbal anticancer composition making granules containing a therapeutically effective amount of active ingredient of plants in an amount of 50 mg to 300 mg and pharmaceutically acceptable carrier(s) comprising MCCP IP (240.0 mg to 290.0 mg), Pregelatinized starch IP (6.0 mg), Colloidal silicon dioxide IP/USP (2.0 mg) and Magnesium stearate IP (2.0 mg) and DM Water (qs) per 50 to 300 mg capsule dosage form.

In yet another preferred embodiment, there is provided a herbal anticancer composition comprising a potency equivalent to about 2 mg to about 100 mg of #STR#1 for once in two days i.v. administration.

In still another embodiment, there is provided a herbal anticancer composition comprising a therapeutically effective amount of solvent extract of plant Sphaeranthus indicus ranging from 100 mg to about 1000 mg for once in two days oral administration.

In still a preferred embodiment, there is provided a method of treating cancer by administering to a patient a natural anticancer composition comprising a therapeutically effective amount of #STR#1 or a extract of plant Sphaeranthus indicus in a pharmaceutically acceptable carrier(s) or otherwise.

In still another embodiment, there is provided a composition inhibits the growth of cancer cells up to 90% at a concentration ranging from 1-6 μg/ml.

### BRIEF DESCRIPTION OF DRAWINGS

Further objects of the present invention together with additional features contributing thereto and advantages accruing therefrom will be apparent from the description of preferred embodiments of the present invention which are shown in the accompanying drawing figures.

FIG. 1: HPLC Chromatograms of Extract S-1 to S-10

FIG. 2: Effect of HAC-1 on cell proliferation in different cell lines

FIG. 3: Effect of HAC-1 on Comet formation in MCF-7 cells; Panel A: cells before treatment, Panel B-F: cells after incubation with HAC-1 for 24, 36, 48, 60 and 72 h respectively exhibiting longer comet tails suggesting the increased DNA fragmentation with time of incubation

FIG. 4: Effect of HAC-1 on Telomerase activity

FIG. 5: Apoptosis Induced by HAC-1 in HL60 Cells: DNA fragmentation of HL60 cells after exposure to HAC-1 at 1.6 μg/ml. Five micrograms of DNA was loaded into each lane. Lanes 1 and 2, cells before treatment; lanes 4 and 6, cells treated with HAC-1 for 24 and 48 h respectively.

FIG. 6: Apoptosis Induced by HAC-1 in HL60 Cells: Cells before treatment with HAC-1 exhibiting intact DNA material (Left Top panel) and showing fragmentation after treatment for 12, 24 and 48 h respectively (right top, bottom left and right panel)

FIG. 7: Comparative study of tamoxifen and HAC-1 in NMU induced breast tumours in rats.

FIG. 8: Effect of HAC-1 on weight gain in sarcoma bearing mice.

FIG. 9: Sarcoma bearing mice from positive control group.

FIG. 10: Sarcoma bearing mice from HAC-1 (20 mg/kg) treated group.

FIG. 11: Effect of HAC-1 on differential count in sarcoma bearing mice.

### DETAILED DESCRIPTION OF THE INVENTION

The present invention involves the selection and identification of the herbs and obtaining the extract by subjecting the same to solvent extraction. The bioassay guided fractionation of the extract to identify the active compounds and to develop a method of purification process of active compound for example sesquiterpene lactone and a safe pharmaceutical composition for the use in human beings and animals in all types of cancers and other related diseases as an effective chemotherapy or an adjuvant therapy to the existing anticancer drugs.

Sphaeranthus indicus Linn is one of the 6 species of the genus Sphaeranthus from the family Compositae found in India. It is commonly called as Gorakh mundi. It is an aromatic herb, grows up to 30-60 cm. tall, found abundantly in damp situations in the plains all over India, ascending to an altitude of nearly 1,500 m. in the hills, especially as a weed in the rice-fields. Stems are with toothed wings; and leaves are obovate-oblong, serrate. Flowers are white or purple in color.

All parts of the plant find medicinal uses. The juice of the plant is styptic and said to be useful in liver and gastric disorders. The paste of the herb, made with oil, is applied in itch. The powdered seeds and roots are given as an anthelmintic. A decoction of the roots is used in chest-pains, cough, and bowel complaints. The bark mixed with whey, is said to be a useful application in piles. Flowers are credited with alterative, depurative, and tonic properties. Leaf juice is boiled with milk and sugar-candy and prescribed for cough. Antitubercular properties have also been ascribed to the plant (Kirt. & Basu, II, 1347; Rama Rao, 223; Dastur, Medicinal Plants, 219; Chopra, 1958, 601).

The drug may consist of the whole plant or only capitula (inflorescences). It is mostly administered in the form of its steam-distillate. Steam-distillation of fresh flowering herb yields a red viscous essential oil (0.01-0.02%). The major constituents of essential oil are cadinene (15.3%), α-ionone (12.6%), β caryophyllene (7.4), p-methoxycinnamaldehyde (7.4%), eugenol (7.0%), α-phellandrene (7.0%), ocimene (6.1%), citral (5.4%), α-terpinene (2.2%) and an unidentified sesquiterpene (b. p. 127-128° C.) The constituents differed somewhat from those of the oil obtained from Varanasi (0.01%), [α]^{20 °}_{D}+3.5°, which contained methylchavicol, α-ionone, δ-cadinene and p-methoxycinnamaldehyde as the major constituents and ocimene, α-terpinene, citral, geraniol, geranyl acetate, β-ionone, a new sesquiterpene alcohol called sphae-ranthol (C₁₅H₂₆O; b. p. 179-180° C.), two new sesquiterpenes sphaeranthene (b. p. 141-142° C.) and indicusene (b. p. 148-153° C.) and a phenolic ketone (semicarbazone, m. p. 259-261° C.) as the minor constituents.

A bitter alkaloid, sphaeranthine (C₁₃H₁₉NO₅; m.p. 166-168° C., decomp.) has been reported to occur in the plant. Later work has revealed the presence of a glucoside (m.p. 78-79° C.), which, on hydrolysis, yields an alkaloid (m.p. 67-68° C.) (Baslas, Indian J. appl. Chem., 1960, 23, 150; Basu & Lamsal, J. Amer. pharm. As. sci. Edn, 1946, 35, 274).

Capitula contain albumins, a semi drying fatty oil (up to 5%), reducing sugars, tannins, mineral matter, a volatile oil (0.07%) with a characteristic odour and a bitter taste, and a glucoside (C₂₂H₂₆O₁₂; m.p. 148-149° C.). No alkaloid was detected in the inflorescence. The glucoside on hydrolysis gave a water-soluble aglycone, phenolic in nature. β-D-glucoside of β-sitosterol has been isolated from the flower heads [Tiwari, Proc. nat. Acad. Sci. India, 1946, 16A(pt 2 3& 3), 55; Tiwari, ibid., 1963, 33A, 349; Kartar Singh & Tiwari, ibid., 1943, 13A, 88; Gupta, lndian J. Pharm., 1967, 29, 47].

The method of extraction of plant material disclosed herein the present invention wherein employing any organic solvent or a solvent selected from the group comprising n-hexane, heptane, diethyl ether, chloroform, dichloromethane, ethyl acetate, acetone, alcohol and methanol and their extractive yields with respect to active constituents.

In a particular embodiment of the present invention, there is provided a method of obtaining the active compound #STR#1 containing fraction from n-hexane extract of plant Sphaeranthus indicus by subjecting the extract to bioassay-guided fractionation employing methanol to obtain methanol soluble fraction and methanol insoluble fraction.

Further the present invention provides the isolation of each sesquiterpene lactone from methanol soluble fraction by column chromatography over silica gel using n-hexane and ethyl acetate as eluents to yield active sesquiterpene lactone (#STR#1) and its derivates in n-hexane: ethyl acetate (10%), and n-hexane: ethyl acetate (20%) and n-hexane: ethyl acetate (30%) fractions.

In a particular embodiment of the present invention repeated purification and crystallization of sesquiterpene lactone, 7-hydroxyeudesm-4-en-6,12-olide (#STR#1) is performed in hexane: dichloromethane solvent mixture and repeated crystallization in methanol.

In a particular embodiment of the present invention the active compound (#STR-1#) 7-hydroxyeudesm-4en-6,12-olide could be either form of isomer (α and β) or racemic mixture.

In a particular embodiment of the present invention describes a method of obtaining active fraction by solvent fractionation of methanol extract by employing dichloromethane in to dichloromethane soluble fraction (sesquiterpene lactones rich fraction) and dichloromethane insoluble fraction (sesquiterpene lactone glycosides) rich fraction.

In a particular embodiment of the present invention semi purified fraction is obtained by using solvent fractionation of dichloromethane fraction with methanol in to methanol soluble fraction (active fraction) and methanol insoluble fraction (partially active) fractions.

In a more particular embodiment of the present invention, there is provided a method of purification of methanol soluble fraction by column chromatography and repeated crystallization to obtain pure active compound #STR#1 and its derivatives.

The obtained crude extracts of plant material herein described have screened for in vitro anticancer activity against cancer cells such as human breast cancer cells (MCF-7, MDA-468, SK-Br-3), human colon adinocarcinoma cells (Colo320 DM), human acute promylocytic leukemia cells (HL-60), mouse carcinoma cells (Sarcoma 180), mouse melanoma cells (C57) and dichloromethane extract of being most active.

The anticancer agent disclosed herein the present invention is effective against any kind of cancer cell in mammals including human beings for example human breast cancer cells (MCF-7, MDA-468, SK-Br-3), human liver carcinoma cells (Hep-G2), human colon adinocarcinoma cells (Colo320 DM), human acute promylocytic leukemia cells (HL-60), mouse carcinoma cells (Sarcoma 180), mouse melanoma cells (C57) and human cervical cancer cells (He La).

The anticancer agent #STR#1 obtained from the plant Sphaeranthus indicus disclosed herein the present invention is having anticancer properties against breast, cervix, neuroblastoma, colon, liver, skin, ovary, lung and other soft tissue tumors.

The anticancer agent obtained from the plant Sphaeranthus indicus disclosed herein the present invention exhibits anticancer properties against various cancer cell lines selected mainly from the group consisting breast, colon, liver, sarcoma, melanoma and leukemia.

The pharmaceutical composition disclosed herein inhibits the growth of cancer cells of breast up to 70-80% at a concentration ranging from 1-6 μg/ml and the breast cell line is selected from group consisting of MCF-7, MDA-MB-468, SK Br-3.

The pharmaceutical composition disclosed herein inhibits the growth of cancer cells of colon up to 80% at a concentration ranging from 1-6 μg/ml and the cancer cell line of colon is Colo-320DM.

The pharmaceutical composition disclosed herein inhibits the growth of cancer cells of liver up to 80% at a concentration ranging from 1-6 μg/ml and the cell line of liver is Hep-G-2.

The pharmaceutical composition disclosed herein inhibits the growth of cancer cells of soft tissue tumors up to 90% at a concentration ranging from 1-6 μg/ml and the cancer cell line for soft tissues is Sarcoma-180.

The pharmaceutical composition disclosed herein inhibits the growth of cancer cells of melanoma up to 63% at a concentration ranging from 1-6 μg/ml and the cancer cell line for melanoma is C-57.

The pharmaceutical composition disclosed herein inhibits the growth of cancer cells of human acute promyelocytic Leukemia cells up to 85% at a concentration ranging from 1-6 μg/ml and the cancer cell line for human acute promyelocytic Leukemia is HL-60.

In particular embodiment of the present invention, there is provided a method of chemical modifications of sesquiterpene lactone (#STR#1) to obtain many derivatives (Scheme-Ia and Scheme-Ib) and its in vitro anticancer activity against the said cancer cell lines.

In a particular embodiment of the present invention, the mechanism of action of the pharmaceutical composition for anticancer activity is established in cell line model and in the animal model.

The stock solution of the test samples is prepared in Dimethyl Sulphoxide (DMSO) as per the solvent specification. The working solution (10 mg/ml), was prepared in serum free Dulbecco's Modified Eagle's Medium (DMEM) then filtered and sterilized.

In a particular embodiment of the present invention, the efficacy of various extracts and #STR#1 obtained herein the present invention is tested in 7 different cell models such as human breast cancer cells (MCF-7, MDA-468, and SK-Br-3), human liver carcinoma cells (Hep-G2), human colon adinocarcinoma cells (Colo320 D-M), human acute promylocytic leukemia cells (HL-60), mouse sarcoma cells (Sarcoma 180), mouse melanoma cells (C57/B1/6J). Cells were maintained in DMEM, RPMI 1640 (colo320-D-M and HL-60) and Mecoy's medium (SKBr-3) supplemented with 10% Fetal Calf Serum (FCS) and antibiotics (100 IU/ml of penicillin and 100 μg/ml of streptomycin) till they achieved 80% confluence in a humidified atmosphere containing 5% CO₂ at 37° C. The cultures were passaged upon confluence on a regular basis.

The said cells herein were seeded into a 96-well tissue culture plate or a 24-well tissue culture plate (for DNA studies) at a density of 5×10⁴ cells per well and incubated for 24 hours in a humidified atmosphere containing 5% CO₂ at 37° C. The cells were then washed twice with incomplete medium and incubated with various concentrations of extracts and compounds of formula #STR#1 in serum-free DMEM for 24, 48 and 72 hours. Morphological changes in the cells and cell proliferation activity were recorded. Further after removal of the culture supernatants, MTT assay was performed to assess the viability of cells and cell proliferation assay was performed with different solvent extracts of the said plant and compounds of formula #STR#1 also morphological changes in cells were observed under an inverted binocular microscope and the observations were recorded and comet assay was conducted to demonstrate the apoptotic effects of #STR#1 on cancer cells.

In a more particular embodiment, comets shaped cells were counted. The cancer cells were seeded into a 96-well tissue culture plate at a cell density of 5×10⁴ cells per well in DMEM and incubated with different concentrations of plant extract for 24 hours in a humidified atmosphere containing 5% CO₂ at 37° C. The cells were then washed twice with PBS and ielectrophoresed. The procedure in brief, involved exposing the cell pellet to a high alkaline solution in thin layer of low melting agarose on a slide pre-coated with high melting agarose and subjected for electrophoresis by applying 25 amps of current in a high alkaline buffer for 30 minutes under refrigerated condition after allowing for 15 minutes of denaturation in the tank buffer. The slides were washed with 4 mM TRIS buffer solution and stained with ethedium bromide. The slides were immediately examined under a fluorescent microscope for recording the number of comet shaped cells out of 200 cells counted.

Telomerase PCR ELISA technique is used for assessing telomerase activity of cancer cells in the treatment with the extract composition and compound #STR#1 and gelatinase zymography is conducted to demonstrate the effect of #STR#1 on the cell invasion ability of the cancer cells.

In a more particular embodiment, genomic DNA extraction is done for assaying DNA integrity by direct visualization under fluorescent microscope.

In a more particular embodiment, assay for influence of estrogen is performed to assess the hormone dependent or independent activity on cell proliferation.

Further, the present invention is illustrated in a greater detail by way of the following examples. The examples are given herein for illustration of the invention and are not intended to be limiting thereof.

### EXAMPLE 1

Preparation of Extract from Sphaeranthus indicus by Percolation Method

The shade dried flowers and/or influoresence of Sphaeranthus indicus was pulverized to coarse powder and about 1 Kg each of powdered material placed in different flasks and extracted with n-hexane, n-heptane, petroleum-ether (40-60° C.), diethyl ether, dichloromethane, chloroform, ethyl acetate, acetone, methanol and ethyl alcohol at room temperature for 24 h to 48 h. and the plant extractions were filtered and concentrated to dryness on rotatory evaporator or on a steam bath at optimum temperature and under reduced pressure.

### EXAMPLE 2

Preparation of Extract from Sphaeranthus indicus by Hot-Soxhalation Method

The coarse powdered material of flowers and/or influoresence of Sphaeranthus indicus was subjected to soxhalation using solvents n-hexane, n-heptane, petroleum-ether (40-60°-C.), diethyl ether, dichloromethane, chloroform, ethyl acetate, acetone, methanol and ethyl alcohol at optimum temperature and recycled until extraction is completed and then the plant extractions were filtered and concentrated to dryness on rotatory evaporator or on a steam bath at optimum temperature.

All extracts such as n-hexane extract (S-1), n-heptane extract (S-2), petroleum ether extract (40-60° C.) (S-3), diethyl ether extract (S-4), dichloromethane extract (S-5), chloroform extract (S-6), ethyl acetate extract (S-7), acetone extract (S-8), methanol extract (S-9), and ethyl alcohol extract (S-10) prepared from the flowers and/or inflouresence of Sphaeranthus indicus by using percolation method and/or soxhalation method were subjected to HPTLC (High Performance Thin Layer Chromatography) and HPLC (High performance Liquid chromatography) in various mobile phases on precoated TLC plates (Merck) and ODS column for qualitative and quantitative estimation of sesquiterpene lactone compounds. All solvent extracts S-1 to S-10 prepared by both percolation and soxhalation methods were found to be qualitatively and quantitatively similar to each solvent extract respectively. The extractive yields of solvent extracts are summarized in Table-1.

TABLE 1

| S. No | Code No | Nature of Extract | Yield (%) |
|---|---|---|---|
| 1 | S-1 | n-Hexane | 2.43 |
| 2 | S-2 | n-Heptane | 2.00 |
| 3 | S-3 | Petroleum ether | 2.45 |
| 4 | S-4 | Diethyl ether | 1.40 |
| 5 | S-5 | Dichloromethane | 3.87 |
| 6 | S-6 | Chloroform | 3.00 |
| 7 | S-7 | Ethyl acetate | 3.00 |
| 8 | S-8 | Acetone | 3.13 |
| 9 | S-9 | Methanol | 8.50 |
| 10 | S-10 | Ethyl alcohol | 9.00 |

### EXAMPLE-3

Bioactive Guided Fractionation of n-Hexane Extract

The n-hexane extract (1.5 Kg) was subjected to solvent—solvent fractionation using methanol to obtain methanol soluble fraction, MS-1 (650 g) and methanol insoluble fraction, MI-1 (850 g). The methanol soluble fraction was found to be rich of sesquiterpene lactones and in particularly compound #STR#1 by HPLC. The methanol insoluble fraction is in rich of fatty compound esters, hydrocarbons, and monoterpenes.

### EXAMPLE-4

Column Chromatography of Methanol Soluble Fraction, MS-1 of n-Hexane Extract

About 600 g of bioactive fraction, MS-1 was subjected to column chromatography over silica gel (60-120 mesh). The active fraction suspended in 2 L of methanol and slurry was prepared with 1 Kg of silica gel. About of 3 kg of silica gel column was packed in n-hexane. The column was run with 5 L of n-hexane to remove any silica gel impurities. The slurry was then slowly poured on to the silica gel bed and eluted with n-hexane and then n-hexane: ethylacetate (5%), n-hexane: ethylacetate (10%), n-hexane: ethylacetate (15%), n-hexane: ethylacetate (20%), n-hexane: ethylacetate (25%), n-hexane: ethylacetate (30%). About 120 fractions of 500 ml each were collected and pooled accordingly by comparing TLC over precoated silica gel plates (e-Merck) in hexane: ethylacetate as mobile phase. The similar fractions were mixed together to yield compounds B-1 to B-6. The compound B-6 predominantly more in yields (90 g). The purity of B-6 was improved by repeated crystallization in n-hexane and dichloromethane solvent mixture.

### EXAMPLE-5

A Method of Successive Solvent Extraction of Plant Material Sphaeranthus indicus

About 1 Kg of coarse powder of flowers and/or inflorescence of plant Sphaeranthus indicus was subjected to soxhalation with n-hexane and refluxed at optimum temperature until extraction is completed to yield n-hexane extract (CY-1). The residual plant material was successively subjected soxhalation with chloroform (CY-2), dichloromethane (CY-3), ethylacetate (CY-4) and methanol (CY-5). All these extracts were submitted for in vitro anticancer activity. The methanol extract (CY-5) was found to be active and non-toxic while other solvent extracts (CY-1 to CY-4) were also found to be active and associated with minor cell toxicity.

### EXAMPLE-6

### Column Chromatography of Methanol Extract (CY-5)

About 50 g of methanol extract, CY-5 was subjected to column chromatography over silica gel (60-120 mesh) and eluted with n-hexane, n-hexane-dichloromethane (10%, 25%, 50%), dichloromethane, dichloromethane-ethyl acetate (10%, 15%, 25%, 50%), ethyl acetate, ethyl acetate-methanol (10%, 25%, 50%, 75%) and methanol. A total of 75 fractions of 200 ml each were collected and pooled in to a group after analyzing by TLC in hexane: dichloromethane solvent system. A group of fractions from 23-30 eluted with 15% ethyl acetate in dichloromethane were mixed and concentrated to dryness to yield a sesqueterpene lactone and similar to compound B-6. The fractions from 31 to 75 were identified as glycosidic and amino acid type compounds.

### EXAMPLE-7

### Column Chromatography of Direct Methanol Extract (S-9)

A method of purification of sesquiterpene lactones of formula (#STR#1) was established from direct methanol extract without being subjected to prior solvent—solvent fractionation. Approximately 1 Kg of methanol extract was subjected to column chromatography using a column of 80 cms in length and 15 cms of diameter. About 3.5 Kg silica gel was used for the preparation of column bed using n-hexane. The slurry prepared with 1.3 kg of silica gel was carefully poured on to the silica gel bed and run the column with 20 L of n-hexane, 30 L of 10% ethyl acetate-n-hexane, 50% L of 15% ethyl acete-n-hexane and 20 L of 20% ethyl acetate-n-hexane. These fractions were accordingly mixed after subjecting to thin layer chromatography over precoated silica gel plates (e-Merck) in n-hexane and ethyl acetate (70:30) solvent system. The fractions eluted with 15% ethyl acetate: n-hexane were mixed and concentrated to dryness to obtain almost single compound (nearly 100 g) similar to compound B-6 (#STR#1) and identified as sesquiterpene lactone. The highest purity was achieved by repeated crystallization in n-hexane and dichloromethane mixture.

### EXAMPLE-8

### Structure Elucidation of Compounds B-1 to B-6

Compound B-1: It was obtained as colourless crystalline solid. M.p. 220° C., It gave positive test for Dragon dorff's reagent and identified as alkaloid. TLC (Precoated Silica gel plates): n-Hexane: Ethyl Acetate (70:30), R_{f}: 0.42. It is an unknown alkaloid.

Compound B-2: It was obtained as white crystalline powder, m.p. 80° C., It gave bluish spot on TLC plate (precoated silica gel) after spraying with vanilline (1%) and alcoholic sulphuric acid reagent and heated in hot oven at 100° C. and identified as sesquiterpene lactone. TLC: Mobile Phase: n-Hexane: Ethyl Acetate (70-30) Rf: 0.26. It was identified as frullanolide based on NMR and Mass spectral studies.

Compound B-3: It was obtained as white crystalline powder. m.p. 76° C. It gave blue spot when sprayed with 1% alcoholic sulphuric acid over precoated TLC plates in n-Hexane: Ethyl acetate (70:30) Rf: 0.12. It was identified as 7-hydroxy methyl frullanolide based on NMR and Mass spectral studies

Compound B-4: It was obtained as white crystalline powder, m.p. 80° C., It gave bluish spot on TLC plate (precoated silica gel) after spraying with vanilline (1%) and alcoholic sulphuric acid reagent and heated in hot oven at 100° C. and identified as sesquiterpene lactone. TLC: Mobile Phase: n-Hexane: Ethyl Acetate (70-30) Rf: 0.14 and identified as 2,7 dihydroxy frullanolide based on NMR and Mass spectral studies.

Compound B-5: It was obtained as white crystalline powder, m.p. 71-72° C., [α]_{D}²⁴-64° (c, 3.05) It gave bluish spot on TLC plate (precoated silica gel) after spraying with vanilline (1%) and alcoholic sulphuric acid reagent and heated in hot oven at 100° C. and identified as sesquiterpene lactone. TLC: Mobile Phase: n-Hexane: Ethyl Acetate (70-30) Rf: 0.22

¹H NMR, MS similar to compound B-6.

Compound B-6: It was obtained as white crystalline powder, m.p. 64-65° C., It gave bluish spot on TLC plate (precoated silica gel) after spraying with vanilline (1%) and alcoholic sulphuric acid reagent and heated in hot oven at 100° C. and identified as sesquiterpene lactone. TLC: Mobile Phase: n-Hexane: Ethyl Acetate (80-20) Rf: 0.21

¹H NMR, ¹³C NMR (See Table-2 & 3); mass: m/z 248(M⁺, C₁₅H₂₀O₃), 233 (M⁺-CH₃), 215 (M⁺-CH₃, H₂O), 187, 178, 169, 159, 133, 119, 105, 91, 77, 67, 55, 41. The structure of compound B-6 was tentatively characterized as 7-hydroxy eudesm-4-en-6,12-olide (#STR#1).

TABLE 2

| Position | δH (ppm) | Multiplicity |
|---|---|---|
| H-1α | 1.36 | M |
| H-1β | 1.35 | M |
| H-2α | 1.50 | M |
| H-2β | 1.49 | M |
| H-3β | 2.15 | M |
| H-3α | 1.97 | M |
| H-6 | 5.01 | S |
| H-8α | 1.66 | m |
| H-8β | 1.42 | m |
| H-9α | 1.39 | m |
| H-9β | 1.33 | m |
| H-13α | 5.79 | s |
| H-13β | 6.23 | s |
| H-14 | 1.76 | s |
| H-15 | 1.06 | s |

TABLE 3

| Position | δC (ppm) | Assignment |
|---|---|---|
| C-1 | 38.89 | CH₂ |
| C-2 | 18.25 | CH₂ |
| C-3 | 33.19 | CH₂ |
| C-4 | 140.46 | C |
| C-5 | 127.04 | C |
| C-6 | 81.48 | CH |
| C-7 | 76.05 | C |
| C-8 | 32.81 | CH₂ |
| C-9 | 35.01 | CH₂ |
| C-10 | 31.65 | C |
| C-11 | 144.89 | C |
| C-12 | 169.03 | C |
| C-13 | 120.94 | CH₂ |
| C-14 | 19.41 | CH₃ |
| C-15 | 26.19 | CH₃ |

### EXAMPLE-9

### A Method of HPLC Standardization of Compound B-6 (#STR#1) in Different Solvent Extracts

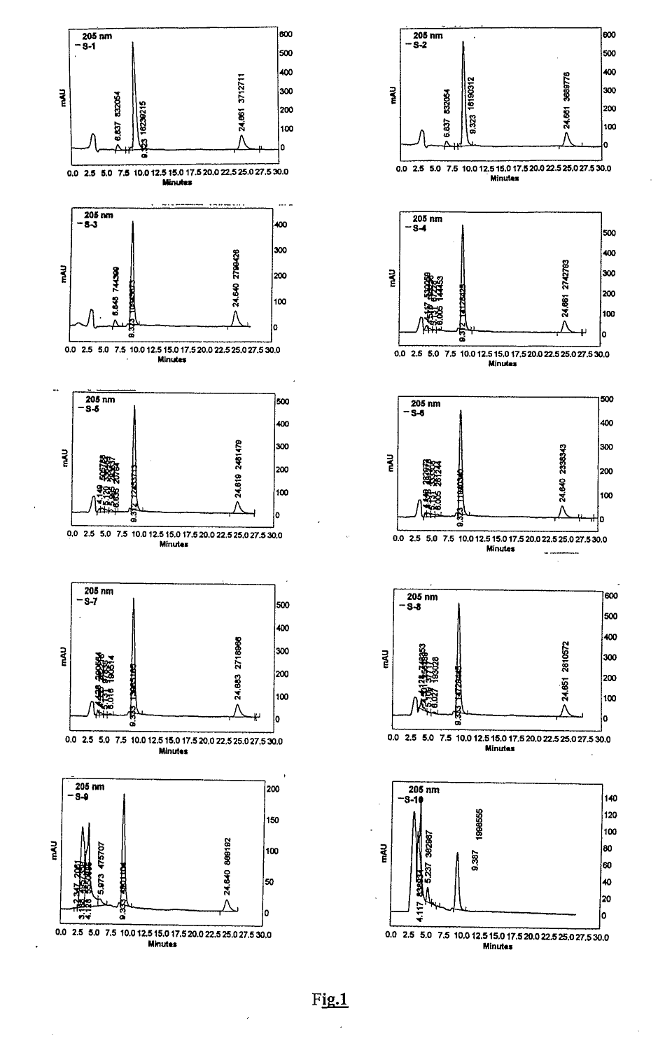

A HPLC method has been developed for qualitative and quantitative estimation of #STR#1 (7-α-hydroxy frullanolide) in different solvent extracts prepared as above. The standard stock solution of #STR#1 of 1 ug/ml solution was prepared in HPLC grade methanol and working standard of 10 ug/ml solution was injected on Thermo Hypersil Keystone C-18 column, (5 u, 250×4.6 mm) and run in mobile phase of 1 part of 0.02% phosphoric acid in double distilled water and 1 part of Acetonitrile at 0.8 ml/min flow rate and the peak was detected at λ205 nm with diode array detector. The solvent extract sample was prepared at 1 mg/ml concentration and analyzed by the above method. The quatitative yields of solvent extracts with respect to compound #STR#1 are summarized in Table-4 and HPLC chromatograms are depicted in FIG. 1.

TABLE 4

| Solvent | Quantity | Yields (#STR#1) |
|---|---|---|
| Extract | (ug/5 mg) | (%)* |
| S-1 | 150 | 0.075 |
| S-2 | 124 | 0.065 |
| S-3 | 123 | 0.062 |
| S-4 | 150.2 | 0.042 |
| S-5 | 127.1 | 0.098 |
| S-6 | 131.9 | 0.078 |
| S-7 | 141.4 | 0.084 |
| S-8 | 149.2 | 0.092 |
| S-9 | 57.63 | 0.091 |
| S-10 | 21.57 | 0.038 |

*On Dry wt basis of plant material.

### EXAMPLE-10

Synthesis of Derivatives of 7-hydroxyeudesm-4-en-6,12-olide (#STR#1)

The compound B-6, 7-hydroxyeudesm-4-en-6,12-olide of formula #STR#1 was subjected to structure activity relationship (SAR) studies to obtain various derivatives of formula #STR#1 as described in Scheme 1A and Scheme 1B.

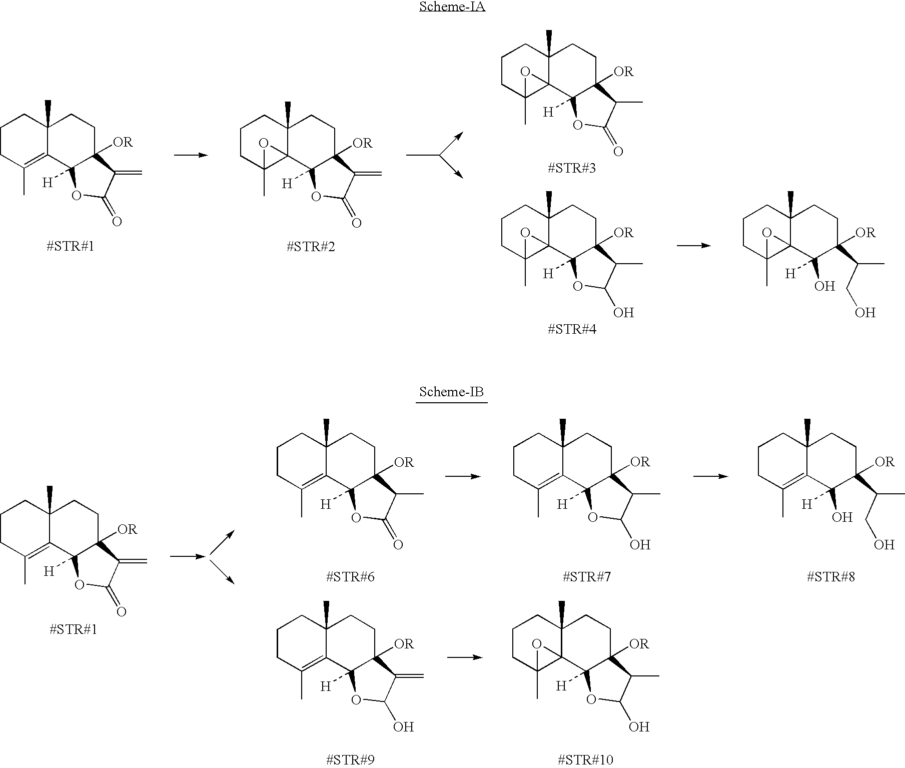

### EXAMPLE-11

Evaluation of Anticancer Activity of Extracts Fractions, Compounds from the Flowers of the Plant

All solvents extracts described above and its fractions, purified compounds including compounds of formula #STR#1 were systematically screened using the following protocol (materials and methods) in vitro, ex-vivo and in-vivo. The highly active compound #STR#1 was coded as HAC-1 for its detailed in vitro studies in various cancer cell lines and in vivo studies in rats.

### Preparation of Sample Solutions

The stock solutions of the samples were prepared in Dimethyl Sulphoxide (DMSO) as per the solvent specification. The working solution (10 mg/ml) was prepared in serum free Dulbecco's Modified Eagle's Medium (DMEM) filter and sterilized.

### Source of Cell Lines

Tumor cells were used for the cell model study to evaluate anticancer properties of the composition. All the cell lines for the present study were procured from National Center for Cell Sciences, Pune and maintained as per the specific requirement for each cell line.

### Screening of Samples

The screening of samples was done in human breast cancer cells (MCF-7, MDA-MB-468, ZR-75-1 and SK-Br-3), human liver carcinoma cells (HepG2), human colon adinocacinoma cells (Colo320 D-M), human acute promylocytic leukemia cells (HL-60), mouse sarcoma cells (Sarcoma 180), mouse melanoma cells (C57/B1/6J). Cells were maintained in DMEM, RPMI 1640(colo320-D-M and HL-60) and Mecoy's medium (SKBr-3) supplemented with 10% Fetal Calf Serum (FCS) and antibiotics (100 IU/ml of penicillin and 100 μg/ml of streptomycin) till they achieved 80% confluence in a humidified atmosphere containing 5% CO₂ at 37° C. The cultures were passaged upon confluence on a regular basis. For assaying the effect of the extract, cells were seeded into a 96-well tissue culture plate or a 24-well tissue culture plate (for DNA studies) at a density of 5×10⁴ cells per well and incubated for 24 hours in a humidified atmosphere containing 5% CO₂ at 37° C. The cells were then washed twice with incomplete medium and incubated with various concentrations of extracts in serum-free DMEM for 24 and 48 h. Morphological changes in the cells were recorded

### Cell Viability Assay

It was performed after removal of the culture supernatants; MTT assay was performed to assess the viability of cells. In brief, 80 μl of serum free medium with 20 μl of MTT (5 mg/ml in phosphate buffered-saline) were added to each well and the plate was incubated at 37° C. for 4 h. Then, 100 μl of 10% sodium dodecyl sulfate (in 0.01 N HCl) was added and the plate was incubated again overnight at 37° C. in a 5% CO₂ incubator to solubilize the formazan crystals. The plates were read on a micro plate reader using a reference wavelength of 690 nm and a test wavelength of 540 nm.

### Cell Proliferation Assay

For assaying the inhibitory effects of the samples on cell proliferation, cancer cells were seeded into a 96-well tissue culture plate at a cell density of 5×10⁴ cells per well and incubated with plant extract at different concentrations for 24 hours in a humidified atmosphere containing 5% CO₂ at 37° C. The viable and dead cell counts were performed as per trypan blue exclusion method and results were recorded.

### Assay for Morphological Changes

For assessing the morphological changes in cells, the cancer cells seeded in a 96 well plate were incubated with serial dilutions of the samples for 24 h in a humidified atmosphere containing 5% CO₂ at 37° C. The morphological changes in the cells were examined under an inverted binocular microscope and the observations were recorded and documented.

### Comet Assay

It was conducted mainly to demonstrate the apoptotic effects of the HAC-1 on the cancer cells. The cancer cells were seeded into a 96-well tissue culture plate at a cell density of 5×10⁴ cells per well in DMEM and incubated with different concentrations of plant extract for 24 hours in a humidified atmosphere containing 5% CO₂ at 37° C. The cells were then washed twice with PBS and subjected to electrophoresis. The procedure in brief, involved exposing the cell pellet to a high alkaline solution in thin layer of low melting agarose on a slide pre-coated with high melting agarose and subjected for electrophoresis by applying 25 amps of current in a high alkaline buffer for 30 minutes under refrigerated condition after allowing for 15 minutes of denaturation in the tank buffer. The slides were washed with 4 mM TRIS buffer solution and stained with ethedium bromide. The slides were immediately examined under a fluorescent microscope for recording the number of comet shaped cells out of 200 cells counted.

### Telo TAGGG Telomerase PCR ELISA

In the present study the telomerase activity of the cancer cell were assessed following the treatment with the samples with the help of Telomerase PCR ELISA (Roche, Germany). Cells were plated in a 24 well plate (2×10⁵ cells per well) in DMEM supplemented with FBS (10%) and incubated for 48 hours and 72 hours in a humidified atmosphere with 95% air and 5% CO₂ at 37° C. with (6.25 μg/ml) or with out (Control) the samples. Cells of the positive control group were treated with paclitaxel at 1.75 μg/ml and incubated as above. This experiment was done in human breast cancer cells (MCF-7). The cell pellets were washed with PBS and resuspended in lysis solution (200 μl) and the debris was pelleted and the supernatant was transferred to a fresh tube. The Telo TAGGG Telomerase PCR ELISA (Roche) was carried as per the manufacturer's protocol. The samples were added to a PCR amplification tube containing reaction mixture (Tris Buffer with telomerase substrate, nucleotides, Taq polymersase biotin labeled primer 1 and primer 2 specific to telomere fragment) and nuclease free water and allowed for elongation for 33 cycles in a Thermal Cycler (MJ Research, PTC 100). The PCR conditions were follows: Cycle 1—10 min 25° C., Cycle 2—5 min 95° C., Cycle 3-32—30 s 94° C.; 30 s 50° C.; 90 s 72° C. and Cycle 33—10 min 72° C. The amplified products were denatured and allowed for hybridization for 2 hours at 37° C. in presence of a hybridization buffer in Microtiter plate (MTP) wells provided in the kit. The MTP well were then washed and treated with anti DIG-POD reagent and incubated for 30 min at 15-25° C. The MTPs were treated with TMB substrate and allowed to react for 10-15 min at 15-25° C. The reaction was stopped with the help of 1N H₂SO₄ and was read in an ELISA plate reader at a test wavelength of 450 nm and reference wavelength of 690 nm.

### Gelatinase Zymography

This study was conducted to demonstrate the effect of HAC-1 on the cell invasion ability of the cancer cells. Before subjecting to the treatment with HAC-1 cells were starved with conditioned media (DMEM+1% bovine Serum Albumin) for 16-18 h in a humidified atmosphere containing 5% CO₂ at 37° C. to assess the levels of Matrix metallo proteinase-9 (MMP-9) which is one of the gelatinases needed for cell invasion. After 24 h of further incubation, cell supernatant were collected. Protein concentrate was prepared using AMICON spin columns and quantified using Bradford's method. Equal amounts of protein were subjected to electrophoresis in a non-reducing gel of strength 7.5% SDS-Polyacrylamide (0.1% gelatin). After the electrophoresis, gels were incubated at 37° C. in zymogram developing buffer for 16 h. Gels were stained with coomassie brilliant blue and the area of clear zones was graded and recorded.

### Genomic DNA Extraction

For assessing the extent of damage to the cellular DNA following the HAC-1 treatment, the cells under investigation were seeded in 25 cm² tissue culture flasks at a cell density of 1×10⁶ cells per flask and incubated for different time points in a humidified atmosphere containing 5% CO₂ at 37° C. The cells after incubation with samples at 7 μg/ml for 24, 48 and 72 hours were pelleted, The cell pelleted were washed with the PBS and incubated in a shaking hot water bath at 37 C. for 1-2 hours with lysis buffer for mammalian DNA extraction. The lysate was then treated with 1.5 volumes of Isopropyl alcohol and the DNA pelleted by centrifuging at 150000 rpm for 20 minutes The DNA was dissolved in Tris EDTA (TE pH 8.00) and was run on 2% agarose and the visualized with the help of a gel documentation system (Pharmacia) and recorded.

### DNA Integrity Assay

It was observed by direct visualization under fluorescent microscope. The cell pellets were treated with acredine orange (t 1:10 dilution) and directly visualized under fluorescent microscope under UV spectrum and the changes in the DNA were documented.

### Assay for Influence of Estrogen

The efficacy of HAC-1 was studied to assess the hormone dependent or independent activity on cell proliferation. For this purpose MCF-7 cells were initially seeded in a 24 well plate with DMEM supplemented with 10% FCS and incubated for 24 hours in a humidified atmosphere with 5% CO₂ and 95% air at 37° C. The cells were then shifted to charcoal striped medium supplemented with 1% bovine albumin serum (BSA,) with or with out the estradiol and HAC-1 and incubated further for a period of 24 h. After the incubation the cell population in each of the groups was recoded by trypan blue exclusion method.

### Statistical Analysis

For calculation of IC₅₀ value, the data were analyzed by no leaner regression (Curve Fit) analysis using Graph Pad Prism version 4.00. The results of cell proliferation and other parameters were analyzed by one-way ANOVA with Bonferroni's Multiple Comparison Test.

### Results

The cell viability assay conducted in samples S-1 to S-10. The most active fraction/extract/HAC-1 was short-listed and taken up for further investigation in other cell models Results are given in Table-5.

TABLE 5
IC₅₀ of plant extracts in various cell lines (in μg/ml)

| | IC₅₀ (in μg/ml) | | | | | | |
|---|---|---|---|---|---|---|---|
| Plant | | | | | | | Colo- |
| Extract | MCF-7 | HL-60 | MDA | C-57 | S-180 | SkBr-3 | 320 |
| S-1 | 31.97 | 5.01 | 30.32 | 26.9 | 41.2 | 31.97 | >60 |
| S-2 | 19.30 | 6.11 | 33.05 | 26.9 | 37.2 | 19.30 | 25.293 |
| S-3 | 30.93 | 5.741 | 24.07 | 36.8 | 39.3 | 30.93 | 28.975 |
| S-4 | 22.64 | 5.53 | 21.35 | 31.5 | 42.9 | 22.64 | 34.697 |
| S-5 | 11.98 | 3.35 | 18.08 | 19.3 | 26.09 | 11.98 | 30.714 |
| S-6 | 16.48 | 5.80 | 14.85 | 36.9 | 53.9 | 16.48 | >60 |
| S-7 | 17.29 | 3.85 | 27.62 | 36.8 | 52.9 | 17.29 | 71.000 |
| S-8 | >60 | 4.28 | 29.03 | 21.9 | 31.9 | >60 | 30.000 |
| S-9 | >60 | 8.87 | >60 | 42.9 | >60 | >60 | >60 |
| S-10 | >60 | 17.60 | >60 | 34.4 | >60 | >60 | >60 |

From the above it is evident that the S-5 plant extract showed lower IC₅₀ values in all the cell lines. However, breast cancer cells required a concentration of 11.98 μg/ml when compared to HL-60 cells that exhibited similar effect at 3.35 μg/ml. Further, the IC₅₀ values of other cells remained at slightly higher levels. Other fractions however generally showed higher IC₅₀ values in all the cell lines.

In the above description, the HPLC standardization of plant extracts and their respective sesquiterpene lactones (of #STR#1) yields revealed that S-5, S-8, S-9, S-1 are respectively high in the yields of compound #STR#1 while the IC₅₀ results demonstrated that S-5 is most active and S-8, S-9 and S-9 are comparatively less active. It was hypothecated that many other compounds present in S-8, S-9, S-1 may interfere the activity of #STR#1 and therefore decided to carry out detailed IC₅₀ studies on compound #STR#1. Many of compounds belongs to the class sesquiterpene lactones isolated and also their derivatives were found be active at more than 200 μg/ml concentration.

The compound HAC-1 (#STR#1) was subjected to in vitro anticancer activity in various cancer cell lines as mentioned above and calculated IC₅₀ values which are summarized in the table-6

TABLE 6
IC₅₀ of HAC-1 in different cell lines

| Cell lines | IC₅₀ (in μg/ml) |
|---|---|
| MCF-7 | 1.62 |
| MDA-MB-468 | 6.83 |
| SK-BR-3 | 0.756 |
| COLO-320-DM | 2.969 |
| HL-60 | 0.468 |
| C-57 | 1.99 |
| S-180 | 7.14 |

### Cell Proliferation Assay of HAC-1

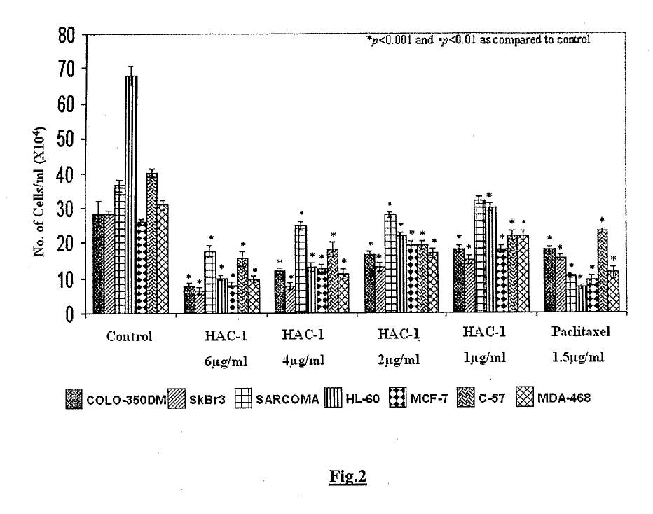

This study was conducted to demonstrate the effect of HAC-1, indicated that the proliferation was significantly inhibited in all the cell lines compared to control. Though all the cells showed significantly lower proliferation compared control (p<0.001 and p<0.01), the HAC-1 concentration for inducing 50% proliferation inhibition varied from 1 to 6 μg/ml. The effective concentration of HAC-1 to induce 50% inhibition was recorded as low as 1 g/ml in HL-60 and SkBr3 cells while it was 2 μg/ml in Colo-320 DM and MDA cells. MCF-7 showed a moderate concentration of 4 μg/ml while Sarcoma cells required 6 μg/ml concentration of HAC-1 to elucidate similar effect (p<0.001). However the results were comparable to the activity of other anticancer drugs. The results of cell proliferation experiment are presented in FIG. 2.

### Morphological Changes

Morphological changes in the tumor cells were observed subsequent to incubation with the HAC-1 were described in the following table-7: The composition showed changes in all the cell lines studied without being specific to mammary cell only.

TABLE 7

| Observation | Control | Treated |
|---|---|---|
| Appearance | Rounded, viable and shining | Irregular and shrunken |
| Adherence | Firm and complete | Incomplete and loose |
| Cell Density | Very High | Very low |
| Viability | Highly viable | Less viable |
| Drug Withdrawl | Cells retain replication ability | Cells loose replication |
| effect | | ability |

### Comet Assay

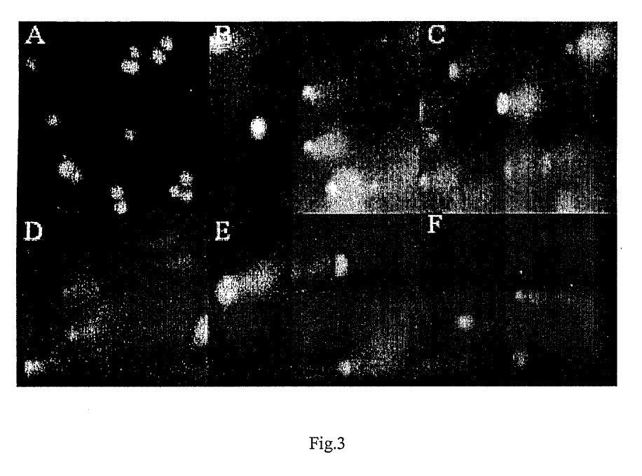

Assay was carried out in various cancer cells treated with the HAC-1 and subsequently run on comet assay protocol indicated that the number of comets in the HAC-1 treated groups were significantly higher (82.5 to 88%) compared to the control cells (8.5%). Results are summarized in Table-8 and FIG. 3

TABLE 8
Effect of HAC-1 on comet formation in cancer cells

| | Number of Cells counted | | |
|---|---|---|---|
| Cell Lines | Non-comet cells | Comets | Percentage of comets |
| MCF-7 | 29 | 171 | 86.5 |
| MDA | 33 | 167 | 83.5 |
| ZR-75 | 27 | 173 | 86.5 |
| SKBr-3 | 35 | 165 | 82.5 |
| Colo-320 DM | 26 | 174 | 87.0 |
| Sarcoma | 28 | 172 | 86.0 |
| HL-60 | 24 | 176 | 88.0 |
| Control | 183 | 17 | 8.5 |

### Telomerase Activity

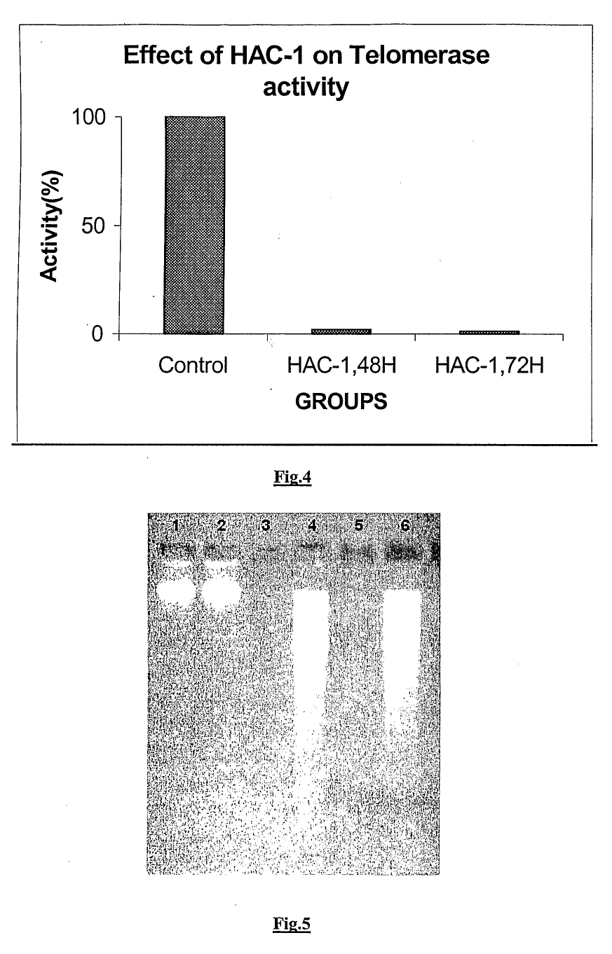

The intactness of telomere fragment in the cancer cells tested by Telo PCR ELISA indicated inhibition in the telomarase activity in the HAC-1 treated group compared to control The result indicates that the HAC-1 has direct inhibitory activity on the telomarase enzyme responsible for retention of telomere fragment as shown in FIG. 4

### Gelatinase Zymography

It was conducted to demonstrate the effect of HAC-1 on the cell invasion ability of the cancer cells with respect to MMP 9 activity showed that the composition inhibits the gelatinase enzyme secretion by the cancer cells. Thus gelatinase zymography indicated that low level of enzyme activity compared to control. The Clear zone produced due to digestion of gelatin was significantly lower in the treated group. Table-9

TABLE 9
Effect of HAC-1 on MMP-9 activity in cancer cells

| Cell lines | MMP-9 Activity |
|---|---|
| MCF-7 | ++ |
| MDA | ++ |
| ZR-75 | + |
| SKBr-3 | ++ |
| COLO-320 DM | ++ |
| HL-60 | + |
| S-180 | ++ |
| Control | ++++ |

++++ Indicates degree of MMP9 activity (Clear zone on the gel)

### Assay for DNA Integrity

This study was conducted to demonstrate the effect of HAC-1 treatment on the tumor cells indicated that incubation for 48 h at a concentration of 7 ug/ml-induced apoptosis. This is evident from the DNA pattern of HL-60 cells following 48 h treatment with HAC-1 presented below. The cells incubated for 24 hours showed lesser degree of apoptosis than the cells incubated for 48 h indicating direct relation with duration of treatment as shown in FIG. 5.

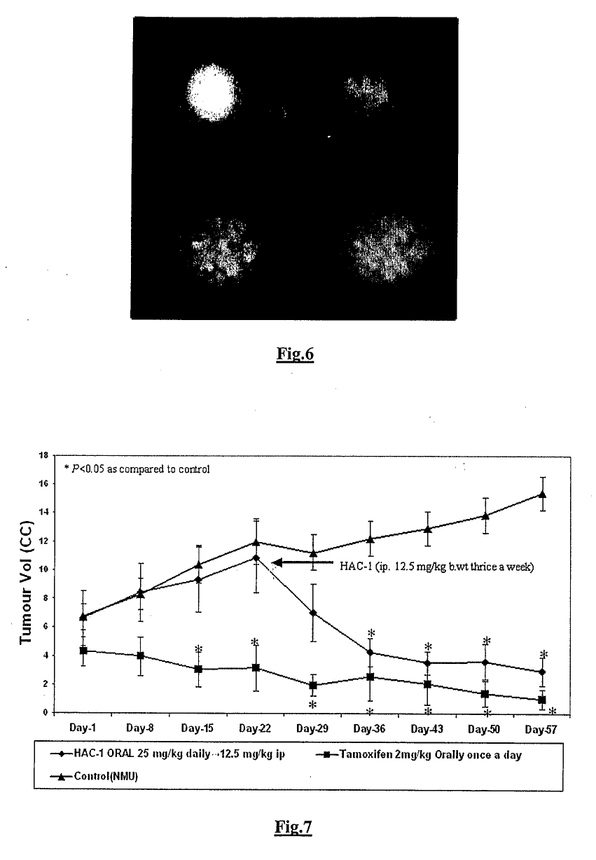

Similarly direct visualization of HAC-1 treated tumor cells (MCF-7) under fluorescent microscope following acredine orange staining indicated the fragmenting DNA material with increasing time of incubation. By the end of 48 h of incubation more than 80% cells were found apoptosed completely. The photographs presented below indicate the DNA pattern before and after the treatment with the drug as shown in FIG. 6

In another experiment the influence of Estrogen was seen. Supplementation of estrogen in the culture medium did not offer protection the tumor cells, indicating that the drug activity is independent of estrogen. Similarly the presence of estrogen in the medium did not influence the dose of drug needed to cause antiproliferative effect. The cell proliferation data remained as presented earlier.

### EXAMPLE-12

Evaluation of Anticancer Activity of HAC-1 by Carcinogen (N-nitroso-N-methylurea) induced Model of Breast Cancer in Rats

Among the multiple experimental animal models employed for analyzing the various aspects of mammary carcinogenesis, the induction of mammary tumors in rats by chemical carcinogens is one of the models most utilized. Experimentally-induced mammary tumors in rodents have proven to constitute useful tools for the study of the pathogenesis of cancer and of the molecular mechanisms involved in the initiation and progression of the neoplastic process. The induction of breast cancer by carcinogen NMU has served as an in vivo model to identify agents that suppress mammary carcinogenesis. By using this model several classes of compounds have been identified that are highly efficacious in inhibiting mammary carcinogenesis. The rat model has advantage over the mouse model, because of the fact that the majority of the mouse lesions are alveolar, while in humans and rats they are predominantly ductal. In rats the most of highly malignant tumours show some common features with intraductal and infiltrating ductal carcinomas in humans. Also the histological structure of rat mammary gland tumours resembles those of human ones.

### Materials and Methods

A standard protocol for induction of breast cancer in female Sprague-Dawley rats (7 to 8 weeks of age and weight range of 80-100 gms) with a single dose of N-nitroso-N-methylurea (NMU) was used for the study. The rats were obtained from animal facility, R & D center, The Himalaya Drug Company, Bangalore. They were maintained on standard pellet diet (Lipton India Ltd., Mumbai) and water ad libitum. Around 60 rats were selected and NMU was administered intraperitoneally at a dose of 50 mg/kg b.wt. NMU was dissolved in saline and pH adjusted to 4.8. Animals were observed daily to assess general health.

### Treatments

One day prior to initiation of treatments, 21 rats bearing tumours of an average volume of 4-6 cm³ were randomly assigned to three groups (with respect to tumour size) 6-8 animals each (12-15 tumors per group).
Group I (n=8), served as control and received vehicle alone for eight weeks.
Group II (n=7) received HAC-1 po daily for three weeks at a dose of 25 mg/kg b.wt. and HAC-1 i.p. for further five weeks at a dose of 12.5 mg/kg b.wt. twice a week.
Group III (n=6). received tamoxifen po daily for eight weeks at a dose of 2 mg/kg b.wt.

### Tumour Measurements

Three longest possible diameters were recorded, and tumour volume (cm³) was calculated using the formula LX W X B.

### Statistical Analysis

The variations of the total surface areas of tumors between day 1 and day 57 were analyzed using a two-way ANOVA for repeated measurements. The treatment effect is thus considered completely confounded with the differences between the groups of animals used within each modality of treatment and is therefore tested against the error term estimated for the animals within the groups. The significance of difference was accepted for p<0.05.

### Results

Intraperitoneal administration of HAC-1 to breast tumor bearing rats, significantly reduced the tumor volume and was comparable to tamoxifen as given in FIG. 7

### EXAMPLE-13

Evaluation of Anticancer Activity of HAC-1 Sarcoma Bearing Mice

Adult, male age-matched Swiss albino mice (29-34 g) housed in groups of four to five in plastic cages, received laboratory rodent chow and tap water ad libitum 14 days prior to experimentation in a temperature controlled room with a 12-h dark/light cycle. S-180 cells maintained in the peritoneal cavity of male mice were used for testing the antitumour activity. The cell suspension was diluted to 1.18×10⁸ cells/ml, and 0.1 ml of the suspension was inoculated intra-peritoneally into the mice. The tumour-bearing mice were treated, 24 hrs after inoculation, by i.p. injections of 10 and 20 mg/kg of HAC-01 (2 mg/ml with vehicle in physiological saline solution) at the dosage of 0.05 and 0.1 ml/10 gm b.wt. of mouse every other day for 14 days (Total 7 injections). The control group was treated with vehicle in 0.9% NaCl. The mice b.wt. was recorded at 0,4,8, 11,16,18,20,22 and 24^{th} day.

### Statistical Analysis

All data were presented as mean ±SD. Statistical significance was evaluated by t test. P<0.05 is considered as statistically significant.

### Hematological Parameters

White blood cell (WBC) and differential leukocyte counts were measured from freely flowing tail vein blood of normal control, sarcoma control and HAC-01 treated groups.

### Results

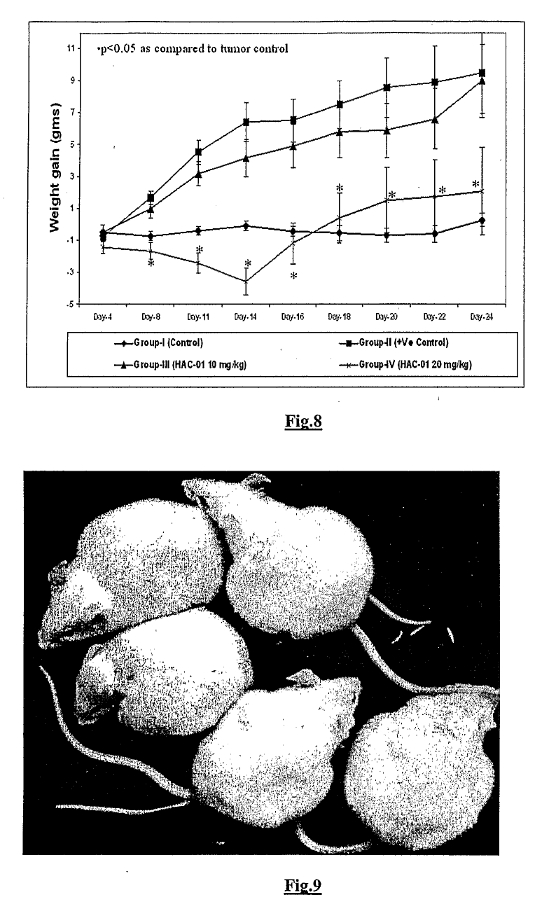

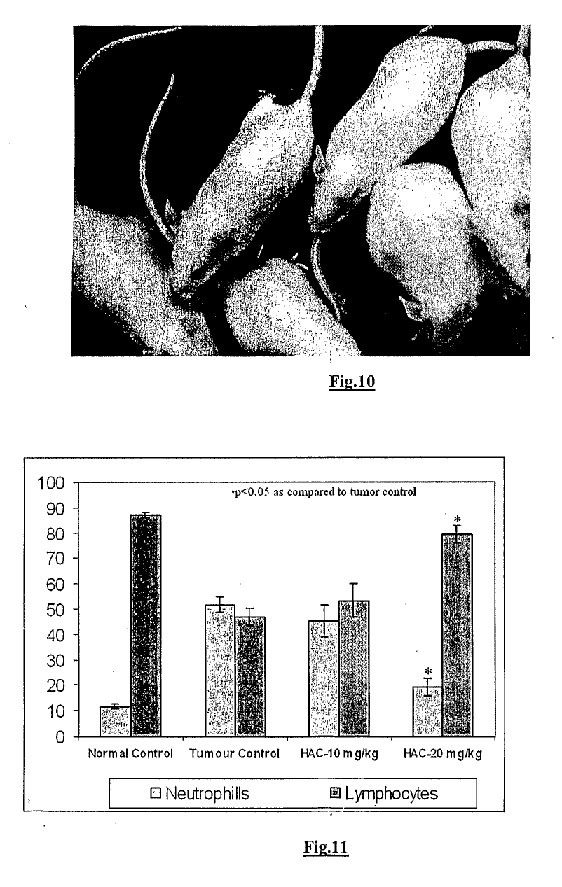

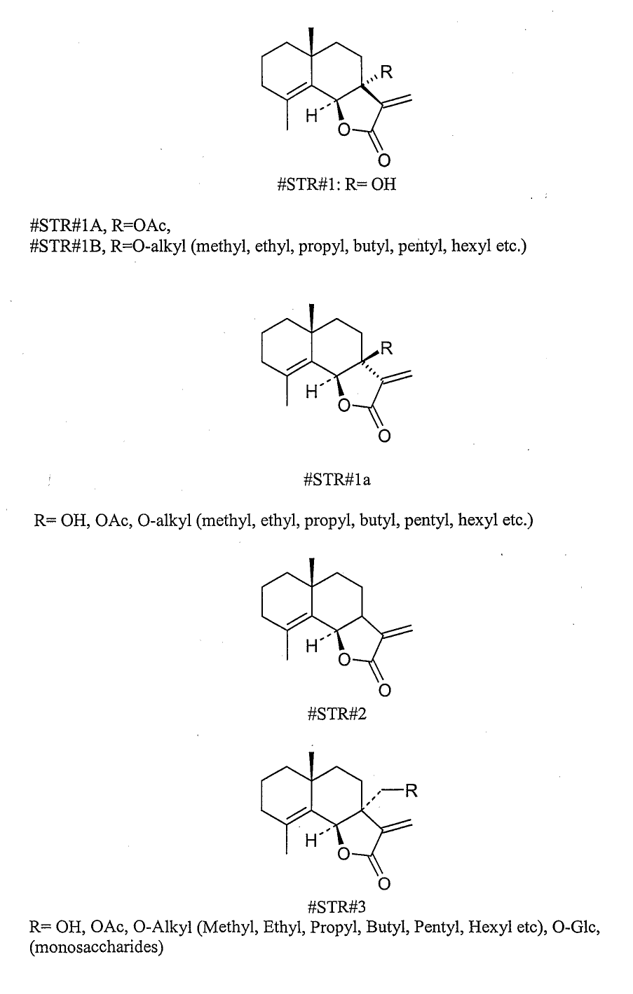

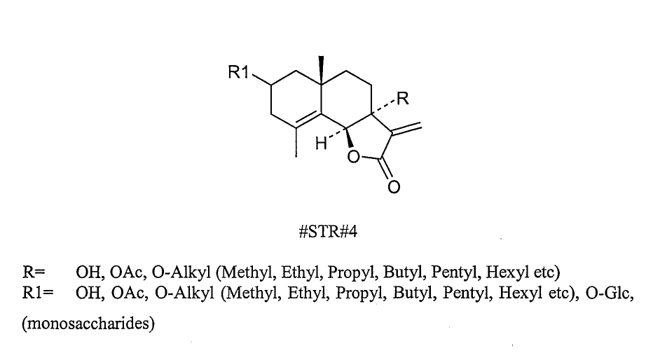

Intraperitoneal administration of HAC-1 to tumour bearing mice significantly reduced the weight gain (FIGS. 8, 9 and 10).

Hematological parameters of tumour-bearing mice showed significant changes when compared with the normal mice. The differential count of WBC showed that the percentage of neutrophils increased (P<0.05) while that of lymphocytes decreased (P<0.05). At the same time interval, HAC-01 (20 mg/kg, i p.) treatment could restore these altered parameters to near normal (FIG. 11).

### EXAMPLE-14

### Preparation of HAC-1 Injections (Formulation I)

Warm the Cremophor ELP to 50° C., and then dissolve HAC-1 into it and then cool to 30° C. Dissolve the solution in Benzyl alcohol and make up to volume with normal saline. Filter the solution through 0.2-micron filter pad. The formulations details for various doses for 1 ml injection are given in Table-11.

TABLE 11

| Sl. No | Name of Ingredient | Formula I | | Formula II | | Formula III | | Formula IV | |
|---|---|---|---|---|---|---|---|---|---|
| 1 | HAC 1 Powder IH | 02.00 | mg | 4.00 | mg | 6.00 | mg | 10.00 | mg |
| 2 | Cremophor ELP* USP NF | 33.33 | mg | 66.66 | mg | 1000.00 | mg | 170.00 | mg |
| 3 | Benzyl alcohol IP | 20.00 | mg | 20.00 | mg | 20.00 | mg | 20.00 | mg |
| 4 | Water for Injection IP | qs to 1 | ml | qs to 1 | ml | qs to 1 | ml | qs to 1 | ml |
| | | Formula V | | Formula VI | | Formula VII | | Formula VIII | |
| 1 | HAC 1 Powder IH | 15.00 | mg | 20.00 | mg | 25.00 | mg | 30.00 | mg |
| 2 | Cremophor ELP* USP NF | 250.00 | mg | 333.33 | mg | 416.00 | mg | 500.00 | mg |
| 3 | Benzyl alcohol IP | 20.00 | mg | 20.00 | mg | 20.00 | mg | 20.00 | mg |
| 4 | Water for Injection IP | qs to 1 | ml | qs to 1 | ml | qs to 1 | ml | qs to 1 | ml |

### EXAMPLE-15

### Preparation of HAC-1 Injections (Formulation II)

Warm the Cremophor ELP to 50° C., and then dissolve HAC-1 into it and then cool to 30° C. Dissolve the solution in Benzyl alcohol and make up to volume with normal saline. Filter the solution through 0.2-micron filter pad. The formulations details for various doses for 1 ml injection are given in Table-12.

TABLE 12

| Sl. No | Name of Ingredient | Formula I | | Formula II | | Formula III | | Formula IV | |
|---|---|---|---|---|---|---|---|---|---|
| 1 | HAC 1 Powder IH | 02.00 | mg | 4.00 | mg | 6.00 | mg | 10.00 | mg |
| 2 | Cremophor ELP* USP NF | 33.33 | mg | 66.66 | mg | 1000.00 | mg | 170.00 | mg |
| 3 | Absolute alcohol IP | qs to 1 | ml | qs to 1 | ml | qs to 1 | ml | qs to 1 | ml |
| | | | | | | Formula | | Formula | |
| | | Formula V | | Formula VI | | VII | | VIII | |
| 1 | HAC 1 Powder IH | 15.00 | mg | 20.00 | mg | 25.00 | mg | 30.00 | mg |
| 2 | Cremophor ELP* USP NF | 250.00 | mg | 333.33 | mg | 416.00 | mg | 500.00 | mg |
| 3 | Absolute alcohol IP | qs to 1 | ml | qs to 1 | ml | qs to 1 | ml | qs to 1 | ml |

### EXAMPLE-16

### Preparation of HAC-1 Tablets

1. Sift Sl. Nos. 1, 2 and 3 through #60 and mix in a suitable mixer for 5 minutes and granulate with DM water to get wet mass. Pass the wet mass through #8 and dry in a suitable dryer at 60-70° C. till the moisture comes to 2 to 4%. Pass the dried granules through #16 and blend the lot uniformly, recheck the moisture, if required redry (moisture content should be 2-4%).
2. Sift Sl. Nos. 4, 5 and 6 through #60 and blend with Step 1 for 10 minutes in a suitable blender.
3. Sift Sl. No. 7 through #60 and blend with Step 2 for 5 minutes in a suitable blender.
4. Compress the tablets as per the specification.

Tablet description: Tablet size: 11 mm round shape, 12.5 mm round shape, 13 mm round shape, 17×8 mm caplet shape and 18×8 mm caplet shape Coating: Uncoated, film coated, sugar coated, enteric coated and sustained release tablet. The details of formulation are summarized in Table-13

TABLE 13

| | | Formula I | Formula II | Formula III | Formula IV |
|---|---|---|---|---|---|
| Sl. No | Name of Ingredient | (mg/tab.) | (mg/tab.) | (mg/tab.) | (mg/tab.) |
| 1. | HAC B1 Powder* IH | 50 | 100 | 150 | 200 |
| 2. | MCCP IP | 224 | 175 | 224 | 274 |
| 3. | Pregelatinized starch IP | 10 | 9 | 10 | 10 |
| 4. | Croscarmellose sodium | 10 | 10 | 10 | 10 |
| | BP | | | | |
| 5. | Crospodione XL USP | 2 | 2 | 2 | 2 |
| 6. | Colloidal silicon dioxide | 2 | 2 | 2 | 2 |
| | IP/USP | | | | |
| 7. | Magnesium stearate IP | 2 | 2 | 2 | 2 |
| | Total weight | 300 | 300 | 400 | 500 |
| | | Formula V | Formula VI | Formula VII | Formula VIII |
| | | (mg/tab.) | (mg/tab.) | (mg/tab.) | (mg/tab.) |
| 8. | HAC B1 Powder* IH | 250 | 300 | 400 | 500 |
| 9. | MCCP IP | 324 | 374 | 474 | 574 |
| 10. | Pregelatinized starch IP | 10 | 10 | 10 | 10 |
| 11. | Croscarmellose sodium | 10 | 10 | 10 | 10 |
| | BP | | | | |
| 12. | Crospodione XL USP | 2 | 2 | 2 | 2 |
| 13. | Colloidal silicon dioxide | 2 | 2 | 2 | 2 |
| | IP/USP | | | | |
| 14. | Magnesium stearate IP | 2 | 2 | 2 | 2 |
| | Total weight | 600 | 700 | 900 | 1100 |

### EXAMPLE-17

### Preparation of HAC-1 Capsules

1. Sift Sl. Nos. 1, 2 and 3 through #60 and mix in a suitable mixer for 5 minutes and granulate with DM water to get wet mass. Pass the wet mass through #8 and dry in a suitable dryer at 60-70° C. till the moisture comes to 2 to 4%. Pass the dried granules through #16 and blend the lot uniformly, recheck the moisture, if required redry (moisture content should be 2-4%).
2. Sift Sl. Nos. 4 and 5 through #60 and blend with Step 1 for 10 minutes in a suitable blender.
3. Fill the capsules as per the specification. Capsule size description: 1, 0 and 00 hard gelatin and vegetable capsules and details are given in Table-14.

TABLE 14

| Sl. | | Formula I | Formula II | Formula III |
|---|---|---|---|---|
| No | Name of Ingredient | (mg/tab.) | (mg/tab.) | (mg/tab.) |
| 1. | HAC B1 Powder* IH | 50 | 100 | 150 |
| 2. | MCCP IP | 240 | 190 | 240 |
| 3. | Pregelatinized starch IP | 6 | 6 | 6 |
| 4. | Colloidal silicon dioxide | 2 | 2 | 2 |
| | IP/USP | | | |
| 5. | Magnesium stearate IP | 2 | 2 | 2 |
| | Total weight | 300 | 300 | 400 |

| Sl. | | Formula IV | Formula V |
|---|---|---|---|
| No | Name of Ingredient | (mg/tab.) | (mg/tab.) |
| 6. | HAC B1 Powder* IH | 200 | 300 |
| 7. | MCCP IP | 285 | 290 |
| 8. | Pregelatinized starch IP | 11 | 6 |
| 9. | Colloidal silicon dioxide IP/USP | 2 | 2 |
| 10. | Magnesium stearate IP | 2 | 2 |
| | | 500 | 600 |

Our detailed studies on anticancer property of HAC-1 (#STR#1) revealed that the cancer cells treated with HAC-1 (#STR#1) had significantly lower cell proliferation rate when compared to control cells. This is an indication of the cell cycle arrest and possibly the effect on cyclin dependent kinase activity. The morphological changes observed in the treated cells indicated that the cells were unable to adhere to the culture plate even at the sub-toxic level suggesting the possibility of cell adhesion molecules being affected with the treatment.

The presence of telomere fragment in the cancer cells is on account of very high telomerase activity, which helps to prevent the DNA damage, and ensures the immortal status to the cell. Significantly lower levels of Telomerase activity observed in the present investigation indicates that the HAC-1 (#STR#1) treated cancer cells were predisposed for high risk of apoptosis. Further, the DNA integrity studies and direct visualization following the incubation with the drug also indicated the damaged and fragmented DNA. The ladder pattern DNA appearance of the HL-60 cells following 48 hours of incubation with the HAC-1 (#STR#1) confirms the pro-apoptotic property of the drug. This was further confirmed with the help comet assay wherein significantly higher levels of comet formations were observed, indicating the intense DNA fragmentation. This was also evident by the fact that the comets exhibited longer tails corresponding to the time of incubation.

Thus these findings indicate that HAC-1 (#STR#1) inhibited cell growth through the induction of apoptosis in tumour cell lines. Further, the anti-proliferative effects were comparable to the effects of paclitaxel. Although paclitaxel exhibited marked growth inhibition at lower doses of 1.75 μg/ml, HAC-1 (#STR#1) was also equally effective at a concentration of 1.62 μg/ml.

In the present investigation, we first showed HAC-1 (#STR#1) exhibited anti-tumour activity in vitro at a concentration of 1.62 μg/ml and that the effect is due to apoptosis in human and other tumour cell lines. The fact that the anticancer activity is not limited to only breast cancer cells, makes this molecule as a broad spectrum chemotherapeutic agent in the treatment of cancerous conditions.

In summary, the present invention provided the first evidence that the HAC-1 (#STR#1) can inhibit the growth of cancer cells by inhibiting telomerase activity and inhibiting the cell invasion capability of the cancer cells. Further, it also induces the apoptosis in the tumour cells. Our findings suggests that the HAC-1 (#STR#1), a plant derived natural compound will provide a novel approach to cancer chemoprevention and/or cancer chemotherapy.

In our experimental studies on rats demonstrated that HAC-1 (#STR#1) is as effective as tamoxifen in reducing tumour volume of breast cancer model in rats when administered by intraperitoneal route. Similarly it is also found to be very effective in sarcoma mouse model as is evident by significant reduction in weight gain.

The present invention also demonstrated the effective treatment for various types of cancers in mammal including human beings. It is also demonstrated the anticancer activity of different solvent extracts and many sesquiterpene lactone type compounds and alkaloids. The present invention is not limited to use of only HAC-1 (#STR#1) as active ingredients in pharmaceutical composition for anticancer chemotherapy. The present invention also mentioned the use of derivatives of #STR#1, solvent extracts, methods of process of extraction of active compounds and their use in any combination and their pharmaceutical dosage forms for treatment and prevention of cancer in human beings.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those of skill in the art without departing from the scope and spirit of this invention.

We claim:

1. A novel herbal anticancer agent comprising the extract of plant Sphaeranthus indicus or group of compounds obtained from the plant Sphaeranthus indicus.

2. The novel herbal anticancer agent according to claim 1, wherein the extract is obtained from all parts of the said plant Sphaeranthus indicus.

3. The novel herbal anticancer agent according to claim 1, wherein the extract is obtained from flowers of the said plant Sphaeranthus indicus.

4. The novel herbal anticancer agent according to claim 1, wherein the extract is obtained from aerial parts of the said plant Sphaeranthus indicus.

5. The novel herbal anticancer agent according to claim 1, wherein the extract is prepared employing an organic solvent alone or mixture thereof.

6. The novel herbal anticancer agent according to claim 5, wherein the organic solvent is selected from hydrocarbons and/or halogenated hydrocarbons and/or alcohol and/or ketone and/or ester and/or ether.

7. The novel herbal anticancer agent according to claim 6, wherein the solvent hydrocarbons is selected from n-hexane, n-heptane, petroleum ether, cyclo hexane etc.

8. The novel herbal anticancer agent according to claim 6, wherein the solvent halogenated hydrocarbon is selected from chloroform, dichloromethane and carbon tetrachloride etc.

9. The novel herbal anticancer agent according to claim 6, wherein the solvent alcohol is selected from methanol, ethanol, propanol and butanol etc.

10. The novel herbal anticancer agent according to claim 6, wherein the solvent ketone is selected from acetone, ethyl methyl ketone etc.

11. The novel herbal anticancer agent according to claim 6, wherein the solvent ester is ethyl acetate.

12. The novel herbal anticancer agent according to claim 6, wherein the solvent ether is diethyl ether.

13. The novel herbal anticancer agent according to claim 1, wherein the extract is prepared by percolation method and/or soxhlation method.

14. The novel herbal anticancer agent according to claim 1, wherein the group of compounds obtained from the plant Sphaeranthus indicus comprises Alkaloids, monoterpenes, sesquiterpenes, sesquiterpene lactones, sesquiterpene, lactone glycosides, diterpenes, triterpenoids, fatty acids esters, hydrocarbons, amino acids.

15. The novel herbal anticancer agent according to claim 1, wherein the group of compounds obtained from the plant Sphaeranthus indicus is preferably sesquiterpenes.

16. The novel herbal anticancer agent according to claim 1, wherein the group of compounds obtained from the plant Sphaeranthus indicus is preferably sesquiterpene lactones.

17. The novel herbal anticancer agent according to claim 16, wherein said sesquiterpene lactone is preferably 7-hydroxyeudesm-4-en-6,12-olide #STR#1 and/or any derivative of the said 7-hydroxyeudesm-4-en-6,12-olide #STR#1.

18. The novel anticancer agent according to claim 17, wherein said 7-hydroxyeudesm-4-en-6,12-olide #STR#1 is in a racemic mixture and/or either form of α or β isomers in pharmaceutical compositions thereof.

19. A herbal composition comprising a therapeutically effective amount of the anticancer agent as claimed in claim 1 and a pharmaceutically acceptable carrier(s) wherein the said composition is effectively used for all types of cancer in mammals including human beings.

20. A delivery system for administering the herbal composition as claimed in claim 19, wherein the delivery system comprises an oral and/or injectable (i.v) form.

21. The delivery system according to claim 20, wherein the delivery system is a tablet.

22. The delivery system according to claim 21, wherein the tablet comprises granules containing a therapeutically effective amount of active ingredient of plant in an amount of 50 mg to 500 mg and pharmaceutically acceptable carrier(s) comprising MCCP IP (224.0 mg to 574.0 mg), Pregelatinized starch IP (10.0 mg), Croscarmellose sodium BP (10.0 mg), Crospodione XL USP (2.0 mg), Colloidal silicon dioxide IP/USP (2.0 mg) and Magnesium stearate IP) (2.0 mg) and DM Water (qs) per 50 to 500 mg tablet dosage form.

23. The delivery system according to claim 20, wherein the delivery system is a capsule.

24. The delivery system according to claim 23, wherein the capsule comprises granules containing a therapeutically effective amount of active ingredient of plants in an amount of 50 mg to 300 mg and pharmaceutically acceptable carriers comprising MCCP IP (240.0 mg to 290.0 mg), Pregelatinized starch IP (6.0 mg), Colloidal silicon dioxide IP/USP (2.0 mg) and Magnesium stearate IP (2.0 mg) and DM Water (qs) per 50 to 300 mg capsule dosage form.

25. The delivery system according to claim 20, wherein the delivery system is in injectable form.

26. The delivery system according to claim 25, wherein the injectable form comprises a therapeutically effective amount of active ingredient of plants in an amount of 2 mg to 30 mg and pharmaceutically acceptable carriers comprising Cremophor ELP (Polyoxyl-35-Casteroil purified) (33.3 mg to 500 mg), Benzyl alcohol (20 mg) and Water for Injection IP (qs) per 1 ml of injectable dosage form.

27. The delivery system according to claim 25, wherein the injectable form comprises a therapeutically effective amount of active ingredient of plants in an amount of 2 mg to 30 mg and pharmaceutically acceptable carriers comprising Cremophor ELP (Polyoxyl-35-Casteroil purified) (33.3 mg to 500 mg), and Absolute alcohol IP (qs) per 1 ml of injectable dosage form.

28. The herbal composition according to claim 19, wherein the composition is capable of being used in the treatment of all type of cancer in mammal including human beings.

29. A method of treating cancer by administering to a patient a herbal anticancer composition comprising a therapeutically effective amount of extract of plant Sphaeranthus indicus or group of compounds obtained from the plant Sphaeranthus indicus in a pharmaceutically acceptable carrier or otherwise.
